(19) United States
(12) Patent Application Publication
(10) Pub. No.: US 2003/0211547 A1
(43) Pub. Date: Nov. 13, 2003

(54) Large conductance calcium-dependent potassium channel as modulator of alcohol effects and consumption

(76) Inventors: Steven L McIntire, Tiburon, CA (US); Andrew G Davies, Berkeley, CA (US); Hongkyun Kim, Emeryville, CA (US)

Correspondence Address: Tom Hunter, Quine Intellectual Property Law Group, P O Box 458, Alameda, CA, 94501, US

(21) Appl. No.: 10/297,269

(22) PCT Filed: Jun. 1, 2001
(86) PCT No.: PCT/US01/17812

(51) Int. Cl. A61K 31/00; G01N 33/53; G01N 33/567; C12Q 1/00

(52) U.S. Cl. 435/7.2; 514/1; 435/4

(57) ABSTRACT

The present invention is directed to the identification of the BK channel as a target for drugs that modulate the effects of ethanol as well as ethanol consumption. The present invention is also directed to the use of modulators of the BK channel to modulate alcohol consumption and the effects of alcohol.

### INTRODUCTION

Field of the Invention

The present invention relates to: screening methods for substances useful for modulating alcohol consumption or altering the effects of alcohol; and the use of modulators of large conductance calcium-activated potassium channels (BK channels) in methods of modulating alcohol consumption and altering the effects of alcohol.

### BACKGROUND OF THE INVENTION

Alcoholism is the most common form of drug abuse and a major public health problem worldwide. The Lewin Group estimated the economic cost to U.S. society in 1992 due to alcohol abuse and alcoholism to be $148 billion (H. Harwood et al., The Economic Costs of Alcohol and Drug Abuse in the United States, 1992, NIH Publication Number 98-4327 (September 1998)). When adjusted for inflation and population growth, the alcohol estimates for 1992 are very similar to cost estimates produced over the past 20 years. The current estimates are significantly greater than the most recent detailed estimates developed for 1985 for alcohol (Rice et al. 1990) and are 42 percent higher for alcohol over and above increases due to population growth and inflation.

Because of the prevalence and societal cost of alcohol abuse and alcoholism, there is a need for drugs that modify alcohol intake or the effects of alcohol on the person consuming it. Few such drugs are currently known.

### SUMMARY OF THE INVENTION

The present invention is directed to assays for identifying substances that modulate alcohol consumption or the effects thereof. The invention encompasses assays for the identification of both compounds acting as BK channel activators and inhibitors, respectively, and the compounds identified by such assays. Such a substance may be identified by comparing BK channel activation in the presence and absence of such substance and by administering such substance to a subject and determining whether the subject experiences enhanced or reduced effects or ethanol or drinks more or less ethanol. Another aspect of the present invention is a kit for identifying substances that modulate activation of the BK channel. Modulation of activation includes both inhibition and activation of the BK channel.

Therapeutically effective amounts of such identified BK channel inhibitors are used for the preparation of pharmaceutical compositions for the modulation of ethanol consumption or the effects of ethanol. In one embodiment, the pharmaceutical compositions comprise a therapeutically effective amount of a compound modulating the activation of the BK channel and a pharmaceutically acceptable carrier. Such compounds can either activate or inhibit the BK channel. These pharmaceutical compositions may be administered to a subject in need for the modulation of ethanol consumption or the effects of ethanol.

In again a further aspect, the present invention relates to the use of pharmaceutical compositions that contain a compound modulating the activation of the BK channel and a pharmaceutically acceptable carrier for the alteration of the effects of ethanol on the recipient or the ethanol consumption of the recipient.

### DETAILED DESCRIPTION OF THE INVENTION

General Overview

The inventors of the present invention have found that C. elegans worms having recessive mutations in the slo-1 gene are more resistant than their wild-type counterparts to the effects of high levels of ethanol on locomotion, egg-laying and feeding behavior. The slo-1 gene encodes the a subunit of a large conductance calcium-activated potassium channel that is highly conserved from worms to mammals to humans. The present invention is accordingly directed to the use of large conductance calcium-activated potassium channels (“BK channels”) as drug targets for the identification of substances that alter the effects of ethanol on the subject exposed to it. Because multiple loss of function mutations in the slo-1 gene cause resistance to the effects of ethanol, the administration of inhibitors of BK channel activation should cause the recipient to be less susceptible to the effects of ethanol. In contrast, subjects that receive enhancers of BK channel activation are expected to experience heightened sensitivity to the effects of ethanol.

Diminished risk of developing alcoholism in humans is associated with increased sensitivity to acute effects of ethanol (M. A. Schuckit, American Journal of Psychiatry 151, 184-18 (1994); T. E. Thiele et al., Nature 396, 366-9 (1998)). Similarly, mutant mice that are more sensitive than wild-type to the acute doses of ethanol have been shown to voluntarily consume less ethanol than their wild-type counterparts. Thus the finding that worms having mutations in the slo-1 gene are less sensitive to acute doses of ethanol than wild-type worms strongly suggests that modulation of BK channel activation will affect ethanol consumption. Although it is believed that increasing a subject's sensitivity to ethanol by increasing activation of the subject's BK channels is likely to cause the subject to decrease its consumption of ethanol, it is possible that decreasing the subject's sensitivity to ethanol by decreasing BK channel activation may decrease ethanol consumption in certain circumstances. For example, a person's desire to imbibe may be attenuated by limiting the person's ability to experience certain effects of ethanol consumption.

BK Channels

Large conductance calcium-activated potassium (slo, maxi-K or BK) channels are potassium-selective, exhibit a very high single-channel conductance (˜100 to 250 pS in symmetric 0.1 M potassium chloride), and are activated by the concerted influences of membrane depolarization and elevated intracellular calcium (Ca⁺⁺) levels (for reviews see Latorre et al., “Molecular workings of large conductance (maxi) calcium-activated potassium channels”, in Handbook of Membrane Channels (Petracchia C, ed.) pp 79-102, Academic Press, San Diego, Calif.; Vergara et a., Current Opinion in Neurobiology 8:321-329 (1998); Sah, Trends in Neuroscience 19:150-154 (1996)). BK channels are found in many cell types, including neurons and smooth muscles. These channels are blocked with some specificity by the venom-derived peptides charybdotoxin and iberiotoxin. All BK channels contain a pore-forming, transmembrane α subunit. The functional channel is believed to be a tetramer of α subunits. In many species, including mammalian species, a β subunit forms a complex with the a subunit and regulates its calcium sensitivity. However, these β subunits are not required for the channel to function. There is no evidence for regulatory β subunits in nematodes or Drosophila.

The gene encoding the BK channel α subunit has been identified in a number of species, including humans (hSlo; Genbank accession nos. U11717, U23767 and U25138), mice (mSlo; Butler, A., et al., Science, 261:221-224 (1993); Genbank accession no. L16912), Drosophila (dSlo “slowpoke” Atkinson et al., Science 253:551-555 (1991); Adelman et al., Neuron 9:209-216 (1992); (Genbank accession no. M69053)), C. elegans (Genbank accession nos. CAB54459 and T27083; Atkinson et al., Drosophila slo locus. Science 1991, 253:551-555; Adelman et al., Neuron 1992, 9:209-216; Jan et al., Annu Rev Neurosci 1997, 20:91-123; Shih et al., J. Cell Biol. 1997, 136:1037-1045; Wei et al., Neuropharmacology 1996, 35:805-829; Meera et al., Proc. Natl. Acad. Sci. USA 1997, 94:14066-14071). The primary sequences of the different mammalian BK channels are almost identical. In many species, the slo transcript is alternatively spliced, giving rise to different variants.

The genome of C. elegans contains two Slo-related genes, slo-1 and slo-2. The C. elegans slo-1 gene encodes an α subunit with 51% sequence similarity with the mouse Slo α subunit mSlo and 60% similarity with the Drosophila α subunit dSlo (Wei et al., 1996 Neuropharmacol. 35(7):805-829). The slo-2 gene encodes a predicted protein that has structural and sequence similarity to the BK channels but that lacks the functional motifs important for calcium and voltage sensing and therefore is likely to have distinct properties (Wei et al. 1996; Lim et al., 1999 Gene 240:35-43).

The fact that mutations affecting the C. elegans BK channel renders the mutant animals resistant to concentrations of ethanol that would paralyze wild-type animals is the first evidence that the BK channel is important in the effects of ethanol in vivo.

Definitions

A “BK channel” means a large conductance, calcium-activated potassium channel. BK channels are also sometimes referred to as: large conductance, calcium-dependent potassium channels; high conductance calcium-activated potassium channels; high conductance calcium-dependent potassium channels; maxi-K channels; and slo channels.

A “pharmaceutically acceptable formulation” comprises a formulation that is suitable for administering the BK channel modulator in a manner that gives the desired results and does not also produce adverse side effects sufficient to convince a physician that the potential harm to a patient is greater than the potential benefit to that patient. The basic ingredient for an injectable formulation is a water vehicle. The water used is of a purity meeting USP standards for sterile water for injection. Aqueous vehicles that are useful include sodium chloride (NaCl) solution, Ringer's solution, NaCl/dextrose solution, and the like. Water-miscible vehicles are also useful to effect full solubility of the BK channel modulator. Antimicrobial agents, buffers and antioxidants are useful, depending on the need.

An “effective amount” is an amount that results in the desired result. Such effective amount will vary from person to person depending on their condition, their height, weight, age, and health, the mode of administering the modulator of BK channel, the particular modulator administered, and other factors. As a result, it is advisable to empirically determine an effective amount for a particular patient under a particular set of circumstances.

A “modulator of BK channel activation” is either an inhibitor of BK channel activation or an enhancer of BK channel activation.

An “inhibitor of BK channel activation” comprises a molecule or group of molecules that (a) interferes with (1) the expression, modification, regulation, activation, degradation, conductance, kinetics or physiology of the BK channel, or (2) the opening of the BK channel, or (b) facilitates the closure of the BK channel. An inhibitor of BK channel activation can also inhibit other potassium channels. However, a selective inhibitor of BK channel activation significantly inhibits BK channel activation at a concentration at which the other potassium channels are not significantly inhibited. An inhibitor “acts directly on the BK channel” when the inhibitor binds to the BK channel via electrostatic or chemical interactions. Such interactions may or may not be mediated by other molecules. An inhibitor acts “indirectly on the BK channel” when its most immediate effect is on a molecule other than the BK channel which influences the expression, activation or functioning of the BK channel or the downstream effects of the BK channel.

An “enhancer of BK channel activation” comprises a molecule or group of molecules that (a) enhances: (1) the expression, modification, regulation, activation or degradation, conductance, kinetics or physiology of the BK channel, or (2) the opening of the BK channel, or (b) inhibits the closure of the BK channel. An enhancer of BK channel activation can also enhance other potassium channels. However, a selective enhancer of BK channel activation significantly enhances one or more normal functions of the BK channel at a concentration at which other potassium channels are not significantly affected. An enhancer “acts directly on the BK channel” when the enhancer binds to the BK channel via electrostatic or chemical interactions. Such interactions may or may not be mediated by other molecules. An enhancer acts “indirectly on the BK channel” when its most immediate effect is on a molecule other than the BK channel which influences the expression, activation or functioning of the BK channel or the downstream effects of the BK channel.

A compound or molecule “modulates activation of the BK channel” if it affects (1) the opening or closing of the BK channel, or (2) the expression, modification, regulation, activation or degradation of the BK channel or a molecule acting upstream of the BK channel in a regulatory or enzymatic pathway.

“Alcohol intoxication” comprises clinically significant maladaptive behavioral or psychological changes (e.g., inappropriate sexual or aggressive behavior, mood lability, impaired judgment, impaired social or occupational functioning) that developed during, or shortly after, alcohol ingestion and accompanied by one (or more) of the following cognitive or coordination effects: (1) slurred speech, (2) incoordination, (3) unsteady gait, (4) nystaginus, (5) impairment in attention or memory, or (6) stupor or coma, wherein the symptoms are not due to a general medical condition or better accounted for by another mental disorder.

The “effects of alcohol” comprises those biochemical or behavioral changes that occur as a result of and within a reasonable time following the consumption of alcohol. Different effects can be expected depending on the amount consumed. For example, the effects of low doses of ethanol include locomotor activation whereas the effects of high doses of ethanol include the symptoms of alcohol intoxication.

The specific items mentioned in the foregoing definitions represent preferred embodiments of the present invention.

Assays for the Identification of Compounds Modulating the Effects of Ethanol or Ethanol Consumption

As elucidated by the present invention, the BK channel is involved in the regulation of ethanol consumption and the effects of ethanol of an animal, including human and non-human mammals. The present invention is based, in part, on the inventors' discovery that slo-1 mutant worms are more resistant to ethanol than wild-type worms. This suggests that the BK channel is a target for the development of drugs that alter the effects or consumption of alcohol. Therefore, modulators of the BK channel activity are useful as agents to down- or up-regulate ethanol consumption and the effects of ethanol in a subject in need, including non-human and human mammals.

In one aspect, the present invention relates to assays for the identification of compounds that modulate and/or interfere with, preferably inhibit, the BK channel activity. Such compounds are useful for the generation of pharmaceutical composition for the modulation of ethanol consumption or the effects of ethanol. Two general types of assays are preferred: electrophysiological and biochemical. Electrophysiological assays may include, for example, patch clamp recordings of currents flowing through large-conductance calcium-activated potassium (maxi-K) channels which can be made from membrane patches excised from cultured bovine aortic smooth muscle cells and other cell types using conventional techniques (Hamill et al., 1981, Pflugers Archiv. 391, 85-100) at room temperature. Glass capillary tubing (GARNER #7052) is pulled in two stages to yield micropipettes with tip diameters of approximately 1-2 microns. Pipettes are typically filled with solutions containing (mM): 150 KCl, 10 HEPES (4-(2-hydroxyethyl)-1-piperazineethanesulfonic acid), 1 Mg, 0.01 Ca⁺⁺, and adjusted to pH 7.20 with 3.7 mM KOH. A high resistance (>10.sup.9 ohms) seal is formed between the sarcolemmal membrane and the pipette, and the pipette is subsequently withdrawn from the cell forming an excised inside-out membrane patch. The patch is excised into a bath solution containing (mM): 150 KCl, 10 HEPES, 5 EGTA (ethylene glycol bis(β-aminoethylether)-N,N,N′,N′-tetraacetic acid), sufficient Ca⁺⁺ to yield a free Ca⁺⁺ concentration of 1-5 μM, and the pH is adjusted to 7.2 with 10.5 KOH. An Axopatch 1C amplifier (Axon Instruments, Foster City, Calif.) with a CV-4 headstage can be used to control the voltage and to measure the currents flowing across the membrane patch. The input to the headstage is connected to the pipette solution with a Ag/AgCl wire, and the amplifier ground is connected to the bath solution with a Ag/AgCl wire covered with a robe filled with agar dissolved in 0.2M KCl. Maxi-K channels are identified by their large single channel conductance (about 250 pS) and sensitivity of channel open probability to membrane potential and intracellular calcium concentration. Compounds may then be screened for an effect on channel activity and the modulation of channel activity by the presence or absence of alcohol.

Alternatively, experiments can be carried out with reconstituted channels. Planar lipid bilayers can be formed from a solution of 1-palmitoyl-2-oleoylphosphatidyl-ethanolamine (POPE) and 1-palmitoyloleoylphosphatidylcholine (POPC) in a 7/3 molar ratio dissolved in decane (50 mg/ml). This lipid solution can be painted across a small hole (250 micron) separating two aqueous compartments and to form bilayers with capacitances of 200-250 pF. The solution on the side that the membranes are added (cis) contained (mM): 150 KCl, 10 HEPES, 0.01 Ca⁺⁺, 3.7 KOH, pH 7.20. The solution on the other side (trans) contain (mM): 25 KCl, 10 HEPES, 0.01 Ca⁺⁺, 3.7 KOH, pH 7.20. Plasma membrane vesicles purified from bovine aortic smooth muscle or other cell types (Slaughter et al., 1989, Biochemistry 28, 3995-4002) are added to the cis side until channel incorporation occurred. After channel incorporation, the concentration of KCl on the trans side is increased to 150 mM to prevent further channel incorporation. The orientation of maxi-K channels after insertion into the bilayer is determined from the calcium and voltage sensitivity of the channel. Increases in calcium or voltage on the intracellular side lead to increases in channel open probability. An Axopatch 1C with a CV-4B headstage is used to control the membrane potential and record currents flowing across the bilayer. The inputs to the amplifier are connected to Ag/AgCl wires which connect to the two sides of the bilayer chamber through small tubes filled with agar dissolved in 0.2M KCl. Experiments can be done at room temperature with test compounds to determine their effect on reconstituted channel activity and the modulation of channel activity by the presence or absence of alcohol.

For the identification and isolation of compounds modifying, inhibiting or enhancing the function of the BK channel according to the invention, suitable cellular systems expressing the BK channel may also be employed. For example, the ability of compounds to open BK channels and increase whole-cell outward (K⁺) BK-mediated currents may be assessed under voltage-clamp conditions by determining their ability to increase cloned mammalian (mSlo or hSlo) BK-mediated outward current heterologously expressed in Xenopus oocytes (Butler, A., et al., Science, 261:221-224 (1993); and Dworetzky, S. I., et al., Mol. Brain Res., 27: 189-193 (1994)). The two BK constructs represent nearly structurally identical homologous proteins that have proven by others (U.S. Pat. No. 5,972,961) to be pharmacologically identical. To isolate BK current from native (background, non-BK) current, the specific and potent BK channel-blocking toxin iberiotoxin (IBTX) (Galvez, A., et al., J. Biol. Chem., 265:11083-11090 (1990)) can be employed at a supramaximal concentration (50 nM). The relative contribution of BK channels current to total outward current can be determined by subtraction of the current remaining in the presence of IBTX (non-BK current) from the current profiles obtained in all other experimental conditions (control, drug, and wash). Recordings can be accomplished using standard two-electrode voltage clamp techniques (Stuhmer, W., et al., Methods in Enzymology, Vol. 207: 319-339 (1992)); voltage-clamp protocols may consist of 500-750 ms duration step depolarizations from a holding potential of −60 mV to +140 mV in 20 mV steps. The experimental media (modified Barth's solution) may consist of (in mM): NaCl (88), NaHCO₃ (2.4), KCl (1.0), HEPES (10), MgSO₄ (0.82), Ca(NO₃)₂ (0.33), CaCl₂ (0.41); pH 7.5.

Cells in an appropriate assay system expressing the BK channel may be exposed to chemical compounds or compound libraries to identify compounds having the desired modulating effects. One such assay system is described in U.S. Pat. No. 5,637,470. Alternatively, the BK channel may be expressed in suitable expression systems, designed to allow for high-throughput testing of compounds from any source, optionally isolated, to identify molecules binding to or having measurable inhibitory effects on the BK channel.

Substances that alter ethanol-induced activation of BK channels without affecting normal BK channel function are preferred. Such substances might also be identified using a cell culture system or yeast that express the α subunit of BK channel of interest. If such BK channel also has a β subunit associated with it, then the cultured cells or yeast or oocytes could express both subunits. Cells expressing the proteins could be exposed to the substances to be tested in combination with ethanol. Additionally, channel activation will result in an efflux of potassium, which can be measured by means of a potassium-sensitive dye such as PBFI (Mevwis et al., Biophys. J. 68:2469-2473 (1995); Molecular Probes (Oregon) product number P-1265) or by quantitating a charge increase outside the cell. Substances that interfere with the effects of ethanol on BK channel activation will at least partially inhibit the ability of ethanol to cause an efflux of potassium from the BK channel.

Another useful biochemical assay determines whether a test compound can successfully compete with charybdotoxin's binding to the BK channel. The interaction of I¹²⁵ChTX (Charybdotoxin) with bovine aortic sarcolemma membrane vesicles is determined under conditions as described in Vazquez et al., 1989, J. Biol. Chem. 264, 20902-20909. Briefly, sarcolemma membrane vesicles are incubated in 12×75 polystyrene robes with ca. 25 pM I¹²⁵ChTX (2200 Ci/mmol), in the absence or presence of test compound, in a media consisting of 20 mM NaCl, 20 mM Tris-HCl pH 7.4, 0.1% bovine serum albumin, 0.1% digitonin. Nonspecific binding is determined in the presence of 10 nM ChTX. Incubations are carried out at room temperature until ligand binding equilibrium is achieved at ca. 90 min. At the end of the incubation period, samples are diluted with 4 ml ice-cold 100 mM NaCl, 20 mM HEPES-Tris pH 7.4 and filtered through GF/C glass fiber filters that have been presoaked in 0.5% polyethylenimine, Filters are rinsed twice with 4 ml ice-cold quench solution. Radioactivity associated with filters is determined in a gamma counter. Specific binding data in the presence of each compound (difference between total binding and nonspecific binding) is assessed relative to an untreated control. The effects of test compounds on open channel probability can be examined in excised inside-out membrane patches and in lipid bilayers.

Substances that block the action of ethanol on BK channels may also be identified by an in vivo screen for substances that suppress an ethanol-induced phenotype. Such phenotypes include differences in locomotion rate, egg-laying frequency, and pharyngeal pumping (feeding) behavior in C. elegans. If human Slo can substitute for slo-1, then worms can be engineered to express hSlo in a slo-1 mutant background. Such worms would be exposed to ethanol in the presence or absence of test substances and substances that cause increased egg-laying in the presence of ethanol can be further tested for effects on slo-1 function. Use of mammalian BK channel proteins in screening assays is preferred, with mouse being more preferred and human being most preferred.

Nucleotide sequences encoding the BK channel may be used to produce the corresponding purified protein using well-known methods of recombinant DNA technology. Among the many publications that teach methods for the expression of genes after they have been isolated is Gene Expression Technology, Methods and Enzymology, Vol. 185, edited by Goeddel, Academic Press, San Diego, Calif. (1990).

The BK channel may be expressed in a variety of host cells, either prokaryotic or eukaryotic. In many cases, the host cells would be eukaryotic, more preferably the host cells would be mammalian. Host cells may be from species either the same or different than the species from which the nucleic acid sequences encoding the protein identified with the methods of the invention are naturally present, i.e., endogenous. Advantages of producing the BK channel by recombinant DNA technology in cellular expression systems include the development of optimized assay systems for the identification of modulating compounds. Generally, the expression systems of the invention have the advantage that they readily provide a system for the production of large amounts of recombinant proteins. However, under certain circumstances that the skilled artisan will appreciate, alternative expression systems may, in some instances, also prove advantageous for obtaining highly enriched sources of the BK channel for purification and the availability of simplified purification procedures. Methods for recombinant production of proteins are generally very well established in the art, and can be found, among other places in Sambrook et al., Molecular Cloning: A Laboratory Manual, 2nd Ed., Cold Spring Harbor (1989).

In an embodiment of the invention, cells transformed with expression vectors encoding the BK channel are cultured under conditions favoring expression of its gene sequence and the recovery of the recombinantly produced protein from the cell culture. The BK channel protein produced by a recombinant cell may be secreted or may be inserted into the plasma membrane, depending on the particular genetic construction used. Purification steps will depend on the nature of the production and the particular protein produced. Purification methodologies are well established in the art; the skilled artisan will know how to optimize the purification conditions. General protocols of how to optimize the purification conditions for a particular protein can be found, among other places, in Scopes in Protein Purification: Principles and Practice, 1982, Springer-Verlag New York, Heidelberg, Berlin.

In addition to recombinant production, peptide fragments of the BK channel may be produced by direct peptide synthesis using solid-phase techniques. See, Stewart et al., Solid-Phase Peptide Synthesis (1969), W. H. Freeman Co., San Francisco; and Merrifield, 1963, J. Am. Chem. Soc. 85:2149-2154. In vitro polypeptide synthesis may be performed using manual techniques or by automation. Automated synthesis may be achieved, for example, using Applied Biosystems 431A Peptide Synthesizer (Foster City, Calif.) following the instructions provided in the instruction manual supplied by the manufacturer.

In an embodiment of the invention, the BK channel protein and/or cell lines expressing the BK channel protein are used to screen for antibodies, peptides, organic molecules or other ligands that act as agonist or antagonists of BK channel activation. Alternatively, screening of peptide libraries or organic compounds with recombinantly expressed the BK channel or cell lines expressing the BK channel may be useful for identification of therapeutic molecules that function by inhibiting, enhancing, or modifying its activation.

Synthetic compounds, natural products, and other sources of potentially biologically active materials can be screened in a number of ways. The ability of a test compound to inhibit, enhance or modulate the function of the BK channel may be determined with suitable assays measuring its activation. For example, responses such as potassium efflux or membrane hyperpolarization may be determined in in vitro assays or cellular assays as described above. These assays may be performed using conventional techniques developed for these purposes.

Finally, the ability of a test compound to inhibit, enhance or modulate the function of the BK channel will be measured in suitable animal models in vivo. For example, mouse models will be used to monitor the ability of a compound to modulate ethanol consumption or the effects of ethanol.

In an embodiment of the invention, random peptide libraries consisting of all possible combinations of amino acids attached to a solid phase support are used to identify peptides that are able to interfere with the activation of the BK channel. For example, peptides may be identified binding to domain of the BK channel or its regulatory β subunit. Accordingly, the screening of peptide libraries may result in compounds having therapeutic value as they interfere with its activation. Identification of molecules that are able to bind to the BK channel may be accomplished by screening a peptide library with recombinant soluble the BK channel protein. Methods for expression and purification of the BK channel protein and may be used to express recombinant full the BK channel protein or fragments thereof, depending on the functional domains of interest. In order to identify and to isolate the peptide/solid phase support that interacts and forms a complex with the BK channel, it is necessary to label or “tag” the BK channel protein molecule or fragment thereof. For example, the BK channel may be conjugated to enzymes such as alkaline phosphatase or horseradish peroxidase or to other reagents such as fluorescent labels which may include fluorescein isothyiocynate (FITC), phycoerythrin (PE) or rhodamine. Conjugation of any given label to the BK channel may be performed using techniques that are routine in the art.

In addition to using soluble the BK channel molecules or fragments thereof, in another embodiment, peptides that bind to the BK channel may be identified using intact cells. The use of intact cells is preferred. Methods for generating cell lines expressing the BK channel identified with the methods and expression systems of the invention. The cells used in this technique may be either live or fixed cells. The cells are incubated with the random peptide library and will bind to certain peptides in the library. The so-formed complex between the BK channel and the relevant solid phase support/peptide may be isolated by standard methods known in the art, including differential centrifugation. An alternative to whole cell assays is to reconstitute the receptor molecules into liposomes where a label or “tag” can be attached.

In another embodiment, cell lines that express the BK channel or, alternatively, isolated the BK channel, or fragments thereof, are used to screen for molecules that inhibit, enhance, or modulate the BK channel's activation. Such molecules may include small organic or inorganic compounds, or other molecules that effect BK channel activation or that promote or prevent the complex formation with its regulatory subunit. In general, the effect of the compound on BK channel activation is scrutinized, and comparing the activation to that of the BK channel, incubated under same conditions, without the compound, thereby determining whether the compound stimulates or inhibits BK channel activation.

The test compounds employed for such assays are obtained from any commercial source, including Aldrich (1001 West St. Paul Ave., Milwaukee, Wis. 53233), Sigma Chemical (P.O. Box 14508, St. Louis, Mo. 63178), Fluka Cherme AG (Industriestrasse 25, CH-9471 Buchs, Switzerland (Fluka Chemical Corp. 980 South 2nd Street, Ronkonkoma, N.Y. 11779)), Eastman Chemical Company, Fine Chemicals (P.O Box 431, Kingsport, Tenn. 37662), Boehringer Mannheim GmbH (Sandhofer Strasse 116, D-68298 Mannheim), Takasago (4 Volvo Drive, Rockleigh, N.J. 07647), SST Corporation (635 Brighton Road, Clifton, N.J. 07012), Ferro (111 West Irene Road, Zachary, La. 70791), Riedel-deHaen Aktiengesellschaft (P.O. Box D-30918, Seelze, Germany), PPG Industries Inc., Fine Chemicals (One PPG Place, 34th Floor, Pittsburgh, Pa. 15272). Further any kind of natural products may be screened using the assay cascade of the invention, including microbial, fungal or plant extracts.

Although any molecule that modulates BK channel activation is sufficient to decrease alcohol consumption, molecules that selectively inhibit BK channel activation are preferred. Since molecules also capable of inhibiting other potassium channels interfere with the various functions performed by those channels, such nonselective inhibitors of the BK channel, although they modulate alcohol consumption, are likely to have many unwanted side effects.

Use of Modulators of BK Channel Activation to Alter Alcohol Consumption

The compounds identified by the methods of the present invention are modulators of the activity of the BK channel, or, alternatively, modulators of protein/protein interactions regulating the BK channel. Such compounds may be useful for the modulation of ethanol consumption or the effects of ethanol. The inventors of the present invention have discovered that worms with mutant forms of the BK channel are much more resistant to the locomotor and other effects of alcohol than wild-type worms. These data indicate that the BK channel regulates acute behavioral responses to ethanol.

Thus, the present invention includes methods for altering alcohol intake by administering a modulator of the BK channel activity to a person desiring to modify his or her alcohol intake. Preferred embodiments decrease alcohol consumption by alcoholics and persons predisposed to alcoholism. While alcoholics and persons predisposed to alcoholism might receive the BK channel inhibitors on a regular basis, those who wish to alter their alcohol consumption at a particular time might self-administer the BK channel modulators at a time prior to, during or following alcohol ingestion. Preferably, the BK channel modulator would be present in the body at a time during which ethanol is also present and would be administered as part of a pharmaceutically acceptable formulation. In addition, the BK channel modulators can be added directly to alcoholic beverages, and a composition comprising alcohol and a modulator of the BK channel is one embodiment of the invention. Effective doses of such modulators can be established by the methods set forth below. Alternatively, the range of effective doses may be established in mice by monitoring drinking behavior and comparing it with that evidenced by the BK channel mutant mice. Such dosages could then be adjusted to account for differences between mice and the species of the subject to be treated.

Methods of modulating consumption of a drug of abuse that involve the administration of an effective amount of a selective inhibitors or selective enhancer of the BK channel are preferred embodiments. Modulators of the BK channel may be small molecule compounds (that is, compounds with a molecular weight of less than or equal to about 2000 daltons, preferably less than or equal to 1000 daltons and most preferably less than or equal to 500 daltons), peptides, proteins, antibodies or the like. Inhibitors that act directly on the BK channel are preferred.

Known modulators of BK channel activation can be used in the instant invention. The scorpion toxin charybdotoxin binds the a subunit (Hanner et al., Proc. Natl. Acad. Sci. USA 1997, 94:2853-2858) and blocks BK channels. Iberiotoxin (Alamone Labs, Jerusalem, Israel), a specific blocker of BK channels (Galvez, A., et al., J. Biol. Chem., 265: 11083-11090 (1990)), is known to be effective at concentrations of about 10 nM. Tetraethylammonium also blocks BK channel activation. Enhancers of BK channel activation that can be administered include: dehydrosoyasaponin (McManus et al., Neuron 1995, 14:645-650); the sesquiterpene compounds described in U.S. Pat. No. 5,399,587; the anthranilic analogs described in U.S. Pat. No. 6,046,239; the 4-aryl-3-aminoquinoline-2-one derivatives described in U.S. Pat. Nos. 5,922,735 and 5,972,961; the 3-substituted oxindole derivatives described in U.S. Pat. No. 5,602,169; the urea derivatives described in U.S. Pat. No. 5,696,138. Preferred doses of such sesquiterpene compounds for adults are within a range of from about 1 mg to 2000 mg (more preferably 5 mg to 200 mg) which may be given in two to four divided doses. Preferred doses of such 4-aryl-3-aminoquinoline-2-one derivatives are within a range of from about 0.1 μg/kg to 1 mg/kg body weight for intravenous administration. Other inhibitors of BK channel activity include the indole diterpene alkaloid compounds described in U.S. Pat. No. 5,541,208.

A range of synthetic and naturally occurring compounds with BK opening activity have been reported. The avena pyrone extracted from avena sativa-common oats has been identified as a BK channel opener using lipid bi-layer technique (International Patent application WO 93/08800). 6-Bromo-8-(methylamino)-imidazo[1,2-a]pyrazine-2-carbonitrile (SCA-40) has been described as a BK channel opener with very limited electrophysiological experiments (Laurent, F. et al., Br. J. Pharmacol. (1993) 108, 622-626). The flavanoid phloretin has been found to increase the open probability of Ca.sup.2+-activated potassium channels in myelinated nerve fibers of Xenopus laevis using outside-out patches (Koh, D-S., et al., Neuroscience Lett. (1994) 165, 167-170). In European patent application EP 477,819 and corresponding U.S. Pat. No. 5,200,422, a number of benzimidazole derivatives were disclosed as openers of BK channels by using single-channel and whole-cell patch-clamp experiments in aortic smooth muscle cells. Further work was reported by Olesen, et al. in European J. Pharmacol., 251, 53-59 (1994). A number of substituted oxindoles have been disclosed as openers of BK channels by P. Hewawasam, et al., in U.S. Pat. No. 5,565,483. Additional modulators of BK channel activation can be identified using the methods described above. The relevant portions of foregoing patents and publications are hereby incorporated by reference.

Another aspect of the invention is a method of modulating the effects of alcohol on the person drinking it. Since the inventors have shown that the BK channel modulates effects of both low doses and high doses of alcohol, this method can be employed in a variety of ways to potentially alter different effects of alcohol, including effects on motor coordination or sedative effects. In one embodiment, a person who wishes to drink alcohol without becoming drunk could take an inhibitor of BK channel activation. This might enable the person to experience the pleasures (taste, socializing, etc.) of alcohol consumption without fear of reduced capacity to operate machinery, participate in sports, or stay awake. In another embodiment, an individual might wish to experience the effects of alcohol without having to drink large quantities of alcohol. For example, a person could become pleasantly tipsy after just one drink, thereby avoiding the calories, expense, and other negative factors associated with consuming more alcohol. A pregnant woman at risk for premature delivery could experience the early labor forestalling effects of alcohol without exposing her fetus to high levels of alcohol.

Formulations/Route of Administration

The identified compounds can be administered to a human patient alone or in pharmaceutical compositions where they are mixed with suitable carriers or excipient(s) at therapeutically effective doses to modulate the effects of ethanol or the consumption of ethanol. Techniques for formulation and administration of the compounds of the instant application may be found in Remington's Pharmaceutical Sciences, Mack Publishing Co., Easton, Pa., latest edition.

Routes Of Administration. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer a compound of the invention in a local rather than systemic manner, for example, via injection of the compound directly into a desired location, often in a depot, or in a sustained release formulation.

Composition/Formulation. The pharmaceutical compositions of the present invention may be manufactured by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually with a greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for protein stabilization may be employed. The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Effective Dosage. Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms, e.g., the symptom of anxiety, of the subject being treated. Determination of the effective amounts is well within the capability of those skilled in the art, especially in light of the detailed disclosure provided herein.

Modulators of BK channel activation can be administered hourly, several times per day, daily or as often as the person undergoing treatment or that person's physician sees fit. Preferably, the administration interval will be in the range of 8 to 24 hours. The degree to which the patient desires to modulate ethanol consumption or the effects of ethanol can be taken into account when determining appropriate intervals for the BK channel inhibitor treatments. the BK channel inhibitor treatments can continue over the course of several days, one month, several months, one year, several years or the duration of the patient's lifetime. Alternatively, the BK channel inhibitors can be administered on a one-time only basis. Inhibitors of BK channel activation should be administered at levels sufficient to reduce produce the desired effect in the body of the patient. The skilled artisan will appreciate that increasing doses of the BK channel inhibitors should be administered until the patient experiences the desired modulation of symptoms, and larger doses fail to effect more desirable modulation.

Inhibitor dosage will vary according to many parameters, including the nature of the inhibitor and the mode of administration. Daily dosages in the range of 1 μg/kg-400 mg/kg of body weight, preferably 1 μg/kg-75 mg/kg and most preferably 10 μg/kg-20 mg/kg are contemplated for the BK channel inhibitors.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between. LD₅₀ and ED₅₀. Compounds that exhibit high therapeutic indices are preferred.

The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in The Pharmacological Basis of Therapeutics, Ch. 1, p.1).

Dosage amount and interval may be adjusted individually to provide plasma levels of the active moiety which are sufficient to maintain the channel modulating effects, or minimal effective concentration (MEC). The MEC will vary for each compound but can be estimated from in vitro data; e.g., the concentration necessary to achieve 50-90% inhibition of the channel using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

The following examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### EXAMPLES

### Example 1

### Generation and Phenotypic Characterization of Ethanol-Resistant C. elegans Mutants

The following example describes the generation and phenotypic characterization of mutants of the free-living nematode C. elegans that have increased resistance to the effects of ethanol as compared to wild-type C. elegans.

Wild-type C. elegans hermaphrodites of the N2 Bristol strain were exposed to the chemical mutagen ethyl methanesulfonate (EMS) to induce random mutations in the germline. In two independent mutagenesis experiments, animals at the L4 stage of development were incubated in a solution of 47 mM EMS for 4 hours at 20° C. These mutagenized animals were transferred to 14-26 plates (8-15 hermaphrodites per plate) and were allowed to self-fertilize to produce the F1 generation. Animals of the F1 generation were transferred to 100-270 new plates (10 hermaphrodites per plate) and allowed to self-fertilize to produce the F2 generation. In the first experiment, the progeny of 2670 F1 animals was screened and in the second experiment, the progeny of 1000 F1 animals was screened. Thus, a total 7340 haploid genomes were screened.

The F2 population was screened to identify animals that were homozygous for recessive mutations or heterozygous for dominant mutations that alter sensitivity to the effects of ethanol on locomotion. Wild-type C. elegans normally move with a sinusoid pattern of locomotion, but when exposed to high concentrations of ethanol, they display extreme, reversible (after the removal of ethanol) uncoordination and paralysis. Animals that are resistant to the effects of ethanol display the sinusoid pattern of locomotion even in the presence of high concentrations of ethanol. To identify the mutants, animals of the F2 generation were washed from their plates into 1.5 ml tubes, rinsed free of bacteria and incubated in a solution of 400 mM ethanol for 30-50 min. The worms were then transferred to the center of 100 mm petri plates that held media containing 400 mM ethanol and a circle of E. coli (OP50) bacteria on the periphery of the media. At this concentration of ethanol, wild-type animals display severe uncoordination and lethargy and are unable to move efficiently towards the bacteria, which are their food source. Animals that moved to the bacteria quickly and with a wild-type pattern of locomotion were identified as ethanol-resistant candidates.

Twenty-eight ethanol resistant candidates were transferred to individual petri dishes and allowed to reproduce. Their progeny were tested for sensitivity to ethanol as described above. Only three candidate animals had progeny that were clearly resistant to the effects of ethanol on locomotion. The progeny of these three candidate animals were maintained as true-breeding strains named BZ6, BZ7 and BZ8 and kept for further examination. An additional mutant, BZ73, which showed locomotion resistance was isolated from an alternative mutagenesis screen.

These mutants were examined for the effect of ethanol on two behaviors other than locomotion: egg laying and pharyngeal pumping. Wild-type C. elegans lay eggs at a reproducible frequency in the presence of their bacterial food source, but high doses (e.g. 400 mM exogenous concentration) of ethanol almost completely suppress egg-laying. Unlike wild-type worms, the egg-laying behavior of BZ6, BZ7 and BZ73 worms was unaffected by high doses of ethanol. In contrast, BZ8 did not display this ethanol-resistance. When exposed to low doses (e.g. 50-100 mM exogenous concentration) of ethanol, BZ6, BZ7 and BZ73 worms lay more eggs than wild-type worms.

Each of these mutants was examined for abnormal phenotypes in the absence of ethanol. The mutants exhibited an obvious defect in locomotion: they displayed an increase in the amplitude of the sinusoidal pattern of locomotion, a phenotype termed loopy locomotion.

Pharmacological analysis of the mutant animals using the acetylcholinesterase inhibitor aldicarb revealed that each of these mutants is hypersensitive to aldicarb. The mutants displayed paralysis at concentrations of aldicarb (0.1-0.25 mM) that only mildly affect wild-type animals. Based on studies of other known mutants in C. elegans, hypersensitivity to aldicarb may indicate an increase in synaptic transmission of acetylcholine compared with wild-type animals. Alternatively, the hypersensitivity to aldicarb may indicate that the mutant's acetylcholine receptors have increased sensitivity. The nature of the hypersensitivity was investigated by treating the animals with the acetylcholine receptor agonist levamisole. BZ6, BZ7 and BZ73 showed a wild-type response to levamisole at concentrations of 60-100 μM, whereas BZ8 appeared hypersensitive to levamisole. The results of this analysis suggest that BZ8 has acetylcholine receptors with increased sensitivity whereas BZ6, BZ7 and BZ73 are likely to have enhanced release of acetycholine into the synapse.

### Example 2

### Genetic and Molecular Analysis of Ethanol Resistance Mutants

This example describes the molecular basis of three of the ethanol-resistant mutant strains described in Example 1. As described below in greater detail, strains BZ6, BZ7 and BZ73 were found to have mutations in slo-1, the gene that encodes the large conductance, calcium-dependent potassium ion channel alpha subunit protein SLO-1.

Complementation analysis was performed on the four ethanol-resistant strains using a genetically marked version of the BZ6 strain. The four mutations strains separated into two complementation groups, one that contained BZ6, BZ7 and BZ73 and one that contained only BZ8. In addition, the mutations were mapped genetically using known Tc1 polymorphisms between the N2 Bristol strain and the RW7000 strain (Williams et al. 1992, Genetics 131:609-624). Tc1 elements are C. elegans transposable elements and the strain RW7000 contains many more Tc1 elements than the N2 Bristol strain. BZ6, BZ7 and BZ73 each mapped between polymorphisms stP108 and stP128 on the right arm of chromosome 5. BZ8 maps elsewhere. BZ6 and BZ73 were mapped more finely and shown to be very close to the polymorphism stP105.

The slo-1 gene is located in the region to which the BZ6 and BZ73 mutants mapped. Because mutants of the slo-1 gene reportedly have a loopy locomotion phenotype like that displayed by the ethanol-resistant mutant strains, the slo-1 genes of the BZ6, BZ7 and BZ73 strains were sequenced to see if these strains contain mutations in the slo-1 gene. The sequence of the slo-1 genomic region is included in the sequence of the yeast artificial chromosome Y51A2 (Genbank accession number AL021497), and the predicted protein sequence of the slo-1 gene product has been published as nSLO (Wei et al., Neuropharmacology 35(7): 805-829 (1996)). DNA sequence analysis of ethanol-resistant C. elegans strains BZ6, BZ7 and BZ73 was performed by first isolating the slo-1 DNA from the mutants by PCR methods using oligonucleotide primers that amplify DNA fragments containing individual or multiple exons of the slo-1 gene. The entire coding region and all intron/exon boundaries contained in these PCR fragments were then sequenced using a Beckman CEQ2000 analysis system.

The sequence data demonstrated that ethanol-resistant strains BZ6 and BZ7 each have a G to A point mutation at position 17009 of the Y51A2 sequence (within exon 6 of slo-1). The mutation resulted in a mis-sense mutation in the predicted protein replacing the Glycine at amino acid position 289 with a Glutamic Acid. Ethanol resistant strain BZ73 has a G to A point mutation at position 16999 of the Y51A2 sequence (also within exon 6 of slo-) which resulted in the replacement of the Glutamic Acid at amino position 286 by a Lysine. Both affected amino acids are located in an extracellular portion of the protein that is adjacent to the membrane, a location that is likely to be sensitive to amino acid charge. The fact that both amino acids are conserved between the predicted nematode and human SLO proteins further suggests that they are important for the structure and/or function of the potassium channel.

### Example 3

### Generation of an mSlo-1—Mouse

The specific role of the BK channel in vivo is further investigated by engineering, via techniques conceptually similar to those taught in U.S. Pat. No. 5,464,764, “knock out” mice in which much of the endogenous murine Slo gene (mSlo) coding sequence is deleted, thereby creating mice which are unable to produce functional mSlo protein. In order to produce the mSlo knock out mice, a mSlo targeting construct is generated which is designed to delete a substantial portion of the mSlo coding sequence upon homologous recombination with the endogenous mSlo gene. This construct also contains markers for positive and negative selection (Joyner A. J., ed. Gene Targeting. The Practical Approach Series, ed. Rickwood D. and Flames B. D., 1993, IRI Press: New York). The completed construct is linearized and transfected by electroporation into embryonic stem (ES) cells having a visible marker (Meiner et al., 1996, Proc. Natl. Acad. Sci. USA 93:14041-14046). ES cells containing the disrupted mSlo gene are produced, isolated by culture with selective agents, confirmed by Southern analysis, and microinjected into murine blastocysts (Bradley, 1987, in Teratocarcinomas and Embryonic Stem Cells: A Practical Approach, E. Robertson, ed. (Oxford: IRL Press), pp. 113-151) to yield mice chimeric for cells containing a disrupted mSlo gene. Offspring of the chimeric mice resulting from germline transmission of the ES genome are obtained and animals heterozygous for the disrupted mSlo are identified by the ES-cell line visible marker and confirmed by Southern analysis. In order to assess the role of mSlo in vivo, the animals heterozygous for the mSlo disrupted gene are bred together, producing lifters containing wild-type mice, mice heterozygous for the mSlo mutation and mice homozygous for the mSlo mutation, which are identified by Southern and Western analysis.

### Example 4

### Responses of mSlo-1—Mice to Ethanol

In mice, low doses of acute ethanol produce locomotor activation, whereas high doses result in sedation (D. A. Finn, P. J. Syapin, M. Bejanian, B. L. Jones, R. L. Alkana, Alcoholism, Clinical and Experimental Research, 18, 382-6 (1994); G. D. Frye, G. R. Breese, Psychopharmacology, 75, 372-9 (1981)). The sensitivity of mSlo^{-/-} mice to acute effects of ethanol is tested as follows.

Each mSlo^{-/-} or mSlo^{+/+} mouse is administered an acute intraperitoneal injection of 2 g/kg ethanol (an amount normally sufficient to induce locomotor activation) and its activity is monitored in a Plexiglas chamber located in a sound-attenuating cubicle equipped with an exhaust fan to mask external noise and pulse-modulated infrared photobeams placed on opposite walls to record ambulatory movements. The activity chambers is computer-interfaced for data sampling at 100-millisecond resolution. Horizontal distance traveled (cm) is recorded for 1 hour.

Each mSlo^{-/-} or mSlo^{+/+} mouse is administered an acute intraperitoneal injection of a sedative dose of ethanol (in the range of 3 to 4 g/kg) and is intermittently placed on its back and tested for loss or righting reflex (LORR). LORR is defined as the inability to complete a righting reflex within a 30-s interval. The duration of LORR is defined as the time interval between LORR and the return of the righting reflex.

To test whether mSlo^{-/-}0 mice have altered absorption, distribution, or clearance of ethanol, blood ethanol concentrations at 10-180 min post intraperitoneal administration of 4.0 g/kg ethanol are measured by drawing a 20 μl blood sample from the tail vein. Blood is added to a centrifuge tube containing 1.8 ml trichloroacetic acid solution and mixed by vortexing. Samples are analyzed using the Sigma Alcohol Diagnostic Kit 332 (Sigma, St. Louis, Mo.).

### Example 5

### Alcohol Consumption in mSlo-1—Mice

To determine whether mSlo modulates ethanol consumption, ethanol preference drinking is compared in wild-type mice and mutant mice lacking mSlo. Ethanol preference drinking is examined using a published method (C. Hodge, C. Slawecki, A. Aiken, Alcohol Clin. Exp. Res., 21, 250-260 (1996)) by which mice have continuous access to two drinking bottles, one containing water and the other containing an ascending range of ethanol concentrations. mSlo^{+/+} and mSlo^{-/-} mice are tested in parallel. Following a one-week acclimatization period during which water is the only fluid available, the mice are given a choice between ethanol (2% w/v) and water. Two-bottle drinking sessions are conducted 23 hours per day, 7 days per week. During the course of the exposure period, ethanol concentration is slowly increased from 2.0% to 14%, with the mice having several days of access to each concentration of ethanol. Each day, the mice are weighed and placed in individual holding chambers while the fluids are attached to the home cage. Initial fluid levels are recorded to the nearest milliliter at the beginning and end of 23 hour fluid-access periods. The position (left or right) of each solution is alternated daily to control for side preferences.

Since reductions in ethanol intake may be influenced by a general disruption of appetite or fluid balance, daily body weights and consumatory behavior by mSlo^{-/-} and mSlo^{+/+} mice are measured over a sustained period. Given that differential taste reactivity may also influence ethanol intake, the same mice are tested for saccharin (sweet) and quinine (bitter) intake and preference in an order-balanced experimental design that can detect taste neophobias (J. C. Crabbe, et al., Nature Genetics, 14, 98-101 (1996)). Saccharin sodium salt and quinine hemisulphate salt (Sigma, St. Louis, Mo.) are dissolved in tap water. These solutions are used for their strong tastes, lack of caloric value, and absence of confounding pharmacological effects. A specific decrease in alcohol consumption would suggest a roe of mSlo in determining the degree of alcohol consumption in mammals.

### Example 6

### Effect of BK Channel Inhibitors and Activators on Consumption of Ethanol and Responses to Ethanol Consumption

Wild-type mice are administered iberiotoxin (Alamone Labs, Jerusalem, Israel), a specific blocker of BK channels, or dehydrosoyasaponin, an activator of BK channels. The sensitivity of the mice to ethanol is tested as described in Example 4 and their ethanol preference drinking is examined as described in Example 5. The results obtained from the mice receiving iberiotoxin or dehydrosoyasaponin are compared with the results of untreated wild-type mice. An effect of one of these drugs would add further support for the concept of regulating alcohol consumption in vertebrates by compounds acting on the BK channel.

### Example 7

### Role of slo-1 in The Neural Effects of Ethanol

The slo-1 gene is expressed in most, if not all neurons, and in body wall muscle but not in pharyngeal muscle. We have shown that, in addition to producing resistance to ethanol for locomotion and egg-laying behaviors, loss-of-function mutations in slo-1 cause resistance to the effects of ethanol on the pharyngeal pumping (feeding) behavior. This latter resistance phenotype implies that ethanol acts on the neurons (which express (slo-1) controlling pharyngeal pumping rather than on the pharyngeal muscles (which do not express SLO-1). Further and stronger confirmation that ethanol acts on neurons in a SLO-1-dependent manner comes from experiments where SLO-1 is expressed from a transgene specifically in neurons or in muscle tissue. We utilized two plasmid constructs for these experiments, BK3.1 and BK4.1. BK3.1 consists of the slo-1 cDNA fused with the promoter from the C. elegans synaptobrevin (snb-1) gene that is expressed exclusively in neurons. BK4.1 has the slo-1 cdNA fused with the promoter from the C. elegans body muscle myosin (myo-3) gene, which is expressed exclusively in body muscle. We transformed animals homozygous for a known null mutation of slo-1(js118) with each of these DNA constructs separately where the concentration of the BK3.1 or BK4.1 constructs was 20 ng/μ1 in the injection solution. So that we could identify transformed progeny of the injected animals we coinjected a DNA construct that expresses the green fluorescent protein (GFP) in neurons with BK3.1 and a construct that expresses GFP in body muscle with BK4.1. In this way we could examine the progeny of the injected animals under a UV microscope and any animals showing GFP fluorescence have been transformed with the injected DNA. If transformed animals are picked to individual plates and their progeny examined for the presence of GFP, only the animals that are germ-line transformants will have progeny expressing GFP. We isolated five independent germ-line transformants for BK3.1 and ten independent germ-line transformants for BK4.1. These strains were tested for their sensitivity to ethanol for the locomotion phenotype. slo-1(js118) animals are very resistant to the effects of ethanol on locomotion showing no decrease in speed in the presence of intoxicating doses of ethanol that cause a 60% decrease in speed by wild-type animals, slo-1(js118) animals carrying a BK3.1 transgene, and therefore only have SLO-1 expressed in neurons, show a 70% decrease in speed, similar to the effect seen in wild-type animals. slo-1(js118) animals carrying a BK4.1 transgene, and therefore only express SLO-1 in body muscle, show little or no decrease in speed on the same concentration of ethanol, similar to the effect of ethanol on slo-1(js118) mutants. These data suggest that in order for ethanol to have an effect on locomotion in C. elegans the slo-1 gene must be expressed in neurons and only needs to be expressed in neurons. slo-1 expression in muscle appears to have little effect on the ethanol sensitivity of C. elegans, at least for the behavior of locomotion.

### Example 8

### Specificity of the Effects of slo-1 on Ethanol Responses in C. elegans

Our data suggests that ethanol acts on the C. elegans nervous system to down-regulate neuronal activity as in the human nervous system. Mutations that increase the level of synaptic transmission result in weak resistance to the effects of ethanol suggesting that the decrease in activity caused by ethanol is balanced by an intrinsic hyperactivity in those mutants. To rule out the possibility of hyperactivity contributing to the resistance seen in slo-1, we compared the level of resistance caused by mutations in slo-1 with the resistance induced by mutations in other regulators of neuron activity including loss-of-function mutations in dgk-1 and goa-1 and a gain-of-function mutation in egl-30. Each of these mutants appears phenotypically hyperactive with increased amplitude of body bends and hyperactive egg laying. For the effects of ethanol on locomotion, three slo-1 mutations js118, eg6 and eg73 showed no decrease in speed at doses of ethanol where wild-type animals showed a 60% decrease in speed. egl-30(js126gf) and dgk-1(sy428) each showed a 40% decrease, an indication of the partial resistance of the hyperactive strains. For the effects of ethanol on the behavior of egg laying, the slo-1 mutations eg24 and eg142 were compared with the hyperactive mutants goa-1(n1134) and egl-30(js126gf), slo-1(eg142) showed no decrease in egg laying rate and slo-1(eg24) showed a 12% decrease compared with a 96% decrease in egg laying in the wild-type strain and 72% and 80% decreases in egl-30(jsl26) and goa-1(n1134) respectively. The fact that the slo-1 mutants show little or no response to the inhibition caused by ethanol compared with moderate to large effects on hyperactive mutants suggests that it is not simply hyperexcitability of neurons in the slo-1 mutants that causes ethanol resistance. Instead, these data are consistent with an effect of ethanol directly on SLO-1. When SLO-1 is absent the ethanol was no direct target in the nervous system and its effects are greatly minimized.

### Example 9

### Determination of eg176 Point Mutation

In the course of a genetic screen for mutants that are resistant to the effects of ethanol, animals bearing the eg176 mutation were isolated as being obviously resistant to the effects of ethanol on locomotion, in other words these animals were moving in a relatively normal fashion while other animals were essentially paralyzed. The eg176 mutation was mapped to the region of the slo-1 gene on chromosome V using the polymorphic mapping strategy as previously described in Example 2. The slo-1 gene was sequenced from DNA of the eg176 strain and a single point mutation was identified indicating that eg176 is an allele of slo-1. The point mutation would cause a serine to phenylalanine amino acid substitution at position 315 of the published predicted SLO-1 protein sequence (Wei et al. (1996) Neuropharm-acology 35(7); 805-829). This mutation is within the pore domain of the SLO-1 potassium channel. The eg176 mutation appears to be dominant or semi-dominant for ethanol resistance in contrast to the loss-of-function mutations of slo-1, which are recessive for their effect on ethanol resistance. The hyperactive phenotype of increased amplitude of body bends also appears to be dominant in the eg176 mutant also in contrast to loss-of-function slo-1 mutations. As slo-1(egl76) is dominant but shares the same phenotype as loss-of-function mutations it appears that it is a dominant negative mutation which suggests that this portion of the channel could be an effective target for potential channel inhibitors.

All publications and patent applications mentioned in this specification are herein incorporated by reference to the same extent as if each individual publication or patent application were specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method for identifying a substance that increases an effect of ethanol on a subject, comprising:
(a) exposing one or more test functional large conductance calcium-dependent potassium (BK) channels to a test substance, and
(b) measuring the activation of the test BK channels, whereby an increased amount of activation of the test BK channels compared to the activation of one or more control functional BK channels which have not been exposed to the test substance is indicative of the test substance being a substance that increases the effect of ethanol on a subject.

2. A method for identifying a substance that decreases an effect of ethanol on a subject, comprising:
(a) exposing one or more test functional BK channels to a test substance, and
(b) measuring the activation of the test BK channels in the presence of a test substance, whereby a decreased amount of activation of the test BK channels compared to the activation of one or more control functional BK channels which have not been exposed to the test substance is indicative of the test substance being a substance that decreases the effect of ethanol on a subject.

3. A method for identifying a substance that alters ethanol consumption, comprising:
(a) exposing one or more test functional BK channels to a test substance, and
(b) measuring the activation of the test BK channels, whereby a detectably different amount of activation of the test BK channels compared to the activation of one or more control functional BK channels which have not been exposed to the test substance is indicative of the test substance being a substance that alters ethanol consumption.

4. The method of claims 1, 2 or 3 wherein the BK channels are contained in one or more cells.

5. The method of claim 4 wherein the BK channels are the only potassium channels contained in the cells.

6. The method of claims 1, 2 or 3 wherein the BK channels are contained in one or more lipid bilayers.

7. The method of claim 6 wherein the BK channels are the only potassium channels contained in the lipid bilayers.

8. The method of claims 1, 2 or 3 wherein the BK channels are mammalian BK channels.

9. The method of claim 8 wherein the BK channels are murine BK channels.

10. The method of claim 8 wherein the BK channels are human BK channels.

11. The method of claims 1, 2 or 3 wherein the substance is selected from the group consisting of a small molecular weight compound, a peptide, a protein, and an antibody.

12. The method of claims 1, 2 or 3 wherein the measuring comprises quantifying potassium effluxes.

13. The method of claim 12 wherein the potassium effluxes are quantified by electrophysiological means.

14. The method of claim 12 wherein the potassium effluxes are quantified by a potassium-sensitive stain indicator compound.

15. The method of claim 1 wherein the test substance increases the amount of activation of the test BK channels at a concentration at which it does not detectably affect the activation any potassium channel that is not a BK channel.

16. The method of claims 1, 2 or 3 wherein the effect of ethanol is a locomotor effect.

17. The method of claims 1, 2 or 3 wherein the effect of ethanol is a cognitive function effect or a coordination effect.

18. A method for identifying compounds that enhance an effect of ethanol on a subject, comprising:
(a) exposing a cell which expresses a functional BK channel to a test compound, and
(b) determining whether the test compound increases activation of the BK channel,
in which test compounds that increase activation of the BK channel are identified as compounds for enhancing the effect of ethanol.

19. A method for identifying compounds that inhibit an effect of ethanol on a subject, comprising:
(a) exposing a cell which expresses a functional BK channel to a test compound, and
(b) determining whether the test compound decreases activation of the BK channel,
in which test compounds that decrease activation of the BK channel are identified as compounds for inhibiting the effect of ethanol.

20. A method for identifying compounds that alter ethanol consumption, comprising:
(a) exposing a cell which expresses a functional BK channel to a test compound, and
(b) determining whether the test compound modulates activation of the BK channel,
in which test compounds that modulate the activation the BK channel are identified as compounds for altering ethanol consumption.

21. A method for identifying compounds that increase an effect of ethanol on a subject, comprising:
(a) exposing a cell which expresses a functional BK channel to ethanol in the presence and absence of a test compound, and determining whether the test compound increases the ethanol-induced activation of the BK channel,
(b) administering the test compound to a non-human animal, and determining whether the test compound increases the effect ethanol on the treated animal, wherein test compounds that increase activation of the BK channel and increase the effect of ethanol on the treated animal are identified as compounds for increasing the effects of ethanol.

22. A method for identifying compounds that decrease an effect of ethanol on a subject, comprising:
(a) exposing a cell which expresses a functional BK channel to ethanol in the presence and absence of a test compound, and determining whether the test compound decreases the ethanol-induced activation of the BK channel,
(b) administering the test compound to a non-human animal, and determining whether the test compound decreases the effect ethanol on the treated animal, wherein test compounds that decrease activation of the BK channel and decrease the effect of ethanol on the treated animal are identified as compounds for decreasing the effects of ethanol.

23. A method for identifying compounds that alter ethanol consumption, comprising:
(a) exposing a cell which expresses a functional BK channel to ethanol in the presence and absence of a test compound, and determining whether the test compound modulates the ethanol-induced activation of the BK channel,
(b) administering the test compound to a non-human animal, and determining whether the test compound alters the ethanol consumption of the treated animal, wherein test compounds that modulate activation of the calcium-dependent potassium channel and alter ethanol consumption of the treated non-human animal are identified as compounds for altering ethanol consumption.

24. The method of claim 23 in which the compounds decrease ethanol consumption.

25. A method for identifying compounds that decrease an effect of ethanol on a subject, comprising:
(a) contacting a test compound with a BK channel, and determining whether the test compound interacts with the BK channel,
(b) administering the test compound to a non-human animal, and determining whether the test compound decreases the effect of ethanol on the treated animal, wherein test compounds that interact with the BK channel and decrease the effect of ethanol on the treated animal are identified as compounds that decrease the effect of ethanol on a subject.

26. A method for identifying compounds that increase an effect of ethanol on a subject, comprising:
(a) contacting a test compound with a BK channel, and determining whether the test compound interacts with the BK channel, and
(b) administering the test compound to a non-human animal, and determining whether the test compound increases the effect of ethanol on the treated animal, wherein test compounds that interact with the BK channel and increase the effect of ethanol on the treated animal are identified as compounds that increase the effect of ethanol on a subject.

27. A method for identifying compounds that regulate ethanol consumption, comprising:
(a) contacting a test compound with a BK channel, and determining whether the test compound interacts with the BK channel, and
(b) administering the test compound to a non-human animal, and determining whether the test compound regulates the ethanol consumption of the treated animal, wherein test compounds that interact with the BK channel and regulate ethanol consumption of the treated animal are identified as compounds that regulate ethanol consumption.

28. The method according the claim 25, 26 or 27 in which the BK channel is contained in an isolated membrane or is recombinantly expressed.

29. A pharmaceutical composition comprising a substance identified by the method of claim 1 through 17 and a pharmaceutically acceptable carrier.

30. A pharmaceutical composition comprising a compound identified by the method of claim 18 through 28 and a pharmaceutically acceptable carrier.

31. A method of increasing an effect of ethanol on a subject by administering to the subject an effective amount of a substance identified by the method of claim 1.

32. A method of decreasing an effect of ethanol on a subject by administering to the subject an effective amount of a substance identified by the method of claim 2.

33. A method of altering a subject's consumption of ethanol by administering to the subject an effective amount of a substance identified by the method of claim 3.

34. A method of increasing an effect of ethanol on a subject by administering to the subject an effective amount of a compound identified by the method of claim 18, 21 or 26.

35. A method of decreasing an effect of ethanol on a subject by administering to the subject an effective amount of a compound identified by the method of claim 19, 22 or 25.

36. A method of altering a subject's consumption of ethanol by administering to the subject an effective amount of a compound identified by the method of claim 20, 23 or 27.

37. A method of modulating the ethanol consumption of a subject, said method comprising: administering to the subject an effective amount of a modulator of BK channel activation.

38. The method of claim 37, wherein said modulator is an inhibitor of BK channel activation.

39. The method of claim 38, wherein said modulator is a selective inhibitor of BK channel activation.

40. A method of modulating the effects of ethanol on a subject, said method comprising: administering to the subject, an effective amount of a modulator of BK channel activation.

41. The method of claim 40, wherein said modulator is an inhibitor of BK channel activation and said effects of ethanol are enhanced.

42. The method of claim 40, wherein said modulator is an activator of BK channel activation and said effects of the ethanol are reduced.
